(19) United States
(12) Patent Application Publication
(10) Pub. No.: US 2005/0124016 A1
(43) Pub. Date: Jun. 9, 2005

(54) Antibodies specific for toxic amyloid beta protein oligomers

(75) Inventors: Mary Jo LaDu, Evanston, IL (US); Lester Binder, Grayslake, IL (US); Blaine Stine, Evanston, IL (US); Arlene Manelli, Chicago, IL (US)

Correspondence Address: David A. Casimir;MEDLEN & CARROLL, LLP, Suite 350, 101 Howard Street, San Francisco, CA, 94105, US

(73) Assignee: ENH Research Institute, Evanston, IL (US)

(21) Appl. No.: 10/909,597

(22) Filed: Aug. 2, 2004

Related U.S. Application Data
(60) Provisional application No. 60/491,725, filed on Aug. 1, 2003.

(51) Int. Cl. G01N 33/53; G01N 33/537; G01N 33/543; C12N 5/06

(52) U.S. Cl. 435/7.92; 530/388.22; 435/334

(57) ABSTRACT

The present invention provides compositions and methods for diagnosing Alzheimer's disease (AD). In particular, the present invention provides monoclonal antibodies that specifically bind to soluble, non-fibrillar oligomeric amyloid β protein assemblies proteolytically derived from the transmembrane amyloid precursor protein (APP) while not reacting with fibrillar amyloid β protein assemblies, monoclonal antibodies that specifically bind to fibrillar amyloid β protein assemblies that do not react with soluble, non-fibrillar oligomeric amyloid β protein assemblies, and methods of use of these compositions in the diagnosis of Alzheimer's disease, as well as methods to monitor treatment and/or disease progression of AD in patients.

The present invention claims priority to U.S. Pat. Appln. Ser. No. 60/491,725, filed Aug. 1, 2003, the disclosure of which is herein incorporated by reference in its entirety.

This invention was funded, in part, under NIH Grant AG13854. The government may have certain rights in the invention.

### FIELD OF THE INVENTION

The present invention provides compositions and methods for diagnosing Alzheimer's disease (AD) and other conditions. In particular, the present invention provides monoclonal antibodies that specifically bind to soluble, non-fibrillar oligomeric amyloid β protein assemblies proteolytically derived from the transmembrane amyloid precursor protein (APP) while not reacting with fibrillar amyloid β protein assemblies, monoclonal antibodies that specifically bind to fibrillar amyloid β protein assemblies that do not react with soluble, non-fibrillar oligomeric amyloid β protein assemblies, and methods of use of these compositions in the diagnosis and treatment of Alzheimer's disease, as well as methods to monitor treatment and/or disease progression of AD in patients.

### BACKGROUND OF THE INVENTION

AD is the fourth most common cause of death in the United States, next to heart disease, cancer and stroke. It presently afflicts more than four million people, and this number is expected to double during the next forty years with the aging of the population. AD is also the most common cause of chronic dementia, with approximately two million people in the United States suffering from dementia. At present, it is estimated that ten percent of the population older than 65 years of age have mild to severe dementia. This high prevalence, combined with the rate of growth of the elderly segment of the population, make dementia and particularly AD, important current public health problems.

To date, AD is the third most expensive disease in the United States, and costs approximately $100 billion each year. Costs associated with AD include direct medical costs such as nursing home care, direct non-medical costs such as in-home day care, as well as indirect costs such as lost patient and care-giver productivity. Medical treatment may have economic benefits by slowing the rate of cognitive decline, delaying institutionalization, reducing care-giver hours, and improving quality of life.

AD is a complex multi-genic neurodegenerative disorder characterized by progressive impairments in memory, behavior, language, and visuo-spatial skills, ending ultimately in death. Hallmark pathologies of AD include granulovascular neuronal degeneration, extracellular neuritic plaques with amyloid β protein deposits, intracellular neurofibrillary tangles and neurofibrillary degeneration, synaptic loss, and extensive neuronal cell death. It is now known that these histopathologic lesions of AD correlate with the dementia observed in many elderly people.

Research on the causes of and treatments for AD has led investigators down numerous avenues. Although many models have been proposed, no single model of AD satisfactorily accounts for all neuropathologic findings; nor do these models of AD satisfactorily account for the requirement of aging for disease onset. Cellular changes, leading to neuronal loss and the underlying etiology of the disease, remain unknown. Proposed causes include environmental factors (Perl, Environmental Health Perspective 63:149 [1985]), metal toxicity (Perl et al., Science 208:297 [1980]), defects in beta-amyloid protein metabolism (Shijo et al., Science 258:126 [1992]; and Kosik, Science 256:780 [1992]), and abnormal calcium homeostasis and/or calcium activated kinases (Mattson et al., J. Neuroscience 12:376 [1992]). The mechanisms of disease progression are equally unclear. Considerable human genetic evidence has implicated alterations in production or processing of the human amyloid precursor protein (APP) in the etiology of the disease. However, intensive research has proven that AD is a multifactorial disease with many different, perhaps overlapping, etiologies.

Early detection and identification of AD facilitate prompt, appropriate treatment and care. However, there is currently no laboratory diagnostic test for AD. Although studies have suggested that calcium imaging measurement in fibroblasts were of potential clinical use in diagnosing AD (Peterson et al., Neurobiology of Aging 9:261 [1988]; and Peterson et al., Proc. Natl. Acad. Sci. USA 83:7999 [1986]), other studies using similar cell lines and techniques have shown no difference in calcium levels in Alzheimer's and normal control fibroblasts (Borden et al., Neurobiology of Aging 13:33 [1991]). Thus, there remains a need for diagnostic methods for AD. In particular, reliable and cost-effective methods and compositions are needed to allow reliable diagnosis of AD.

### SUMMARY OF THE INVENTION

The present invention provides compositions and methods for diagnosing Alzheimer's disease and other conditions. In particular, the present invention finds use with any condition (e.g., including but not limited to neurological conditions) that are directly or indirectly linked to the presence or absence of amyloid β protein assemblies. In particular, the present invention provides monoclonal antibodies that specifically bind to soluble, non-fibrillar oligomeric amyloid β protein assemblies proteolytically derived from the transmembrane amyloid precursor protein (APP) while not reacting with fibrillar amyloid β protein assemblies, monoclonal antibodies that specifically bind to fibrillar amyloid β protein assemblies that do not react with soluble, non-fibrillar oligomeric amyloid β protein assemblies, methods of use of these compositions in the diagnosis and treatment of Alzheimer's disease and other conditions, and methods to monitor treatment and/or disease progression of AD in patients, including methods of screening compounds for diagnostic and therapeutic application. For example, the present invention provides antibodies that may be used for therapeutic use through their ability to specifically bind to particular amyloid β protein assemblies. Following binding, the antibody bound complexes may be targeted with therapeutic compounds that are targeted to the complex or can be degraded and/or cleared by endogenous or exogenous routes. Compounds that find use in treating diseases and conditions can be screened for their ability to target, clear, or otherwise interact with amyloid β protein assemblies (e.g., by recognizing or competing with antibody binding). Thus, the present invention provides diagnostic, therapeutic, and drug screening methods related to biological processes that are linked to the presence or absence of specific amyloid β protein assemblies.

In some embodiments, the present invention provides a composition comprising a purified monoclonal antibody that identifies soluble, non-fibrillar oligomeric amyloid β protein assemblies, while not reacting with fibrillar amyloid β protein assemblies. In some embodiments, the soluble, non-fibrillar oligomeric amyloid β protein assemblies comprise 2-12 amyloid β proteins (although the present invention is not limited to any particular size). In some embodiments, the fibrillar amyloid β protein assemblies comprise more than 12 amyloid β proteins. In some embodiments, the amyloid β protein is the β1-42 protein. In still further embodiments, the amyloid β protein assemblies are neurotoxic.

The present invention also provides a hybridoma that secretes a monoclonal antibody that identifies soluble, non-fibrillar oligomeric amyloid β protein assemblies, while not reacting with fibrillar amyloid β protein assemblies. In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins. In some embodiments, the hybridoma secretes a monoclonal antibody that identifies oligomeric amyloid β proteins comprising the β1′-42 protein.

The present invention also provides methods for obtaining and isolating a hybridoma secreting a monoclonal antibody that identifies soluble, non-fibrillar oligomeric amyloid β protein assemblies, while not reacting with fibrillar amyloid β protein assemblies, comprising the steps of: providing spleen cells immunized with an antigen comprising soluble, non-fibrillar oligomeric amyloid β protein assemblies; fusing the immunized cells with myeloma cells under hybridoma-forming conditions; and selecting those hybridomas that secrete monoclonal antibodies that specifically recognize assemblies comprising amyloid β proteins without recognizing fibrillar amyloid β protein assemblies. In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins.

The present invention further provides a method for producing a monoclonal antibody from a hybridoma secreting a monoclonal antibody that identifies soluble, non-fibrillar oligomeric amyloid β protein assemblies, while not reacting with fibrillar amyloid β protein assemblies, comprising the steps of: culturing the hybridoma in an appropriate medium culture and recovering the monoclonal antibody excreted by the hybridoma, or, alternatively, implanting the hybridoma into the peritoneum of a mouse, and, when ascites have been produced by the animal, recovering the monoclonal antibody then formed from the ascites. In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins. In still further embodiments, the present invention provides a monoclonal antibody produced by this method.

In some embodiments, the present invention provides a composition comprising a purified monoclonal antibody that identifies fibrillar amyloid β protein assemblies, while not reacting with soluble, non-fibrillar oligomeric amyloid β protein assemblies. In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins. In some embodiments, the amyloid β protein is the β1-42 protein.

The present invention also provides a hybridoma that secretes a monoclonal antibody that identifies fibrillar amyloid β protein assemblies, while not reacting with soluble, non-fibrillar oligomeric amyloid β protein assemblies. In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins. In some embodiments, the hybridoma secretes a monoclonal antibody that identifies fibrillar amyloid β proteins comprising the β1-42 protein.

The present invention also provides methods for obtaining and isolating a hybridoma secreting a monoclonal antibody that identifies fibrillar amyloid β protein assemblies, while not reacting with soluble, non-fibrillar oligomeric amyloid β protein assemblies, comprising the steps of: providing spleen cells immunized with an antigen comprising fibrillar amyloid β protein assemblies; fusing the immunized cells with myeloma cells under hybridoma-forming conditions; and selecting those hybridomas that secrete monoclonal antibodies that specifically recognize assemblies comprising fibrillar amyloid β protein assemblies, while not reacting with soluble, non-fibrillar oligomeric amyloid β protein assemblies. In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins.

The present invention further provides a method for producing a monoclonal antibody from a hybridoma secreting a monoclonal antibody that identifies fibrillar amyloid β protein assemblies, while not reacting with soluble, non-fibrillar oligomeric amyloid β protein assemblies, comprising the steps of: culturing the hybridoma in an appropriate medium culture and recovering the monoclonal antibody excreted by the hybridoma, or, alternatively, implanting the hybridoma into the peritoneum of a mouse, and, when ascites have been produced by the animal, recovering the monoclonal antibody then formed from the ascites. In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins. In still further embodiments, the present invention provides a monoclonal antibody produced by this method.

The present invention further provides methods for detecting at least one soluble, non-fibrillar oligomeric amyloid β protein assembly, comprising the steps of: providing a sample suspected of containing at least one soluble, non-fibrillar oligomeric amyloid β protein assembly and a monoclonal antibody that identifies soluble, non-fibrillar oligomeric amyloid β protein assemblies, while not reacting with fibrillar amyloid β protein assemblies; contacting the sample with the antibody under conditions such that the antibody binds to the soluble, non-fibrillar oligomeric amyloid β protein assembly, to form an antigen-antibody complex; and detecting the presence of the antigen-antibody complex. In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins. In some embodiments, the sample is selected from the group consisting of blood, plasma, serum, serous fluid, and cerebrospinal fluid. In some preferred embodiments, the sample is from a subject. In particularly preferred embodiments, the subject is a human. In further embodiments, the subject is selected from the group consisting of subjects displaying pathology resulting from Alzheimer's disease, subjects suspected of displaying pathology resulting from Alzheimer's disease, and subjects at risk of displaying pathology resulting from Alzheimer's disease. In some particularly preferred embodiments, the methods further comprise the step of diagnosing Alzheimer's disease. In additional particularly preferred embodiments, the Alzheimer's disease diagnosed using the methods of the present invention is selected from the group consisting of late onset Alzheimer's disease, early onset Alzheimer's disease, familial Alzheimer's disease and sporadic Alzheimer's disease. In some preferred embodiments, the methods further comprise the step of monitoring the efficacy of treatment of Alzheimer's disease.

In some preferred embodiments, the methods comprises an enzyme-linked immunosorbent assay. In particularly preferred embodiments, the enzyme-linked immunosorbent assay is selected from the group consisting of direct enzyme-linked immunosorbent assays, indirect enzyme-linked immunosorbent assays, direct sandwich enzyme-linked immunosorbent assays, indirect sandwich enzyme-linked immunosorbent assays, and competitive enzyme-linked immunosorbent assays. In alternative preferred embodiments, the antibody used in the methods of the present invention further comprises a conjugated enzyme, wherein the conjugated enzyme is selected from the group of enzymes consisting of horseradish peroxidases, alkaline phosphatases, ureases, glucoamylases, and β-galactosidases. In some particularly preferred embodiments, the enzyme-linked immunosorbent assay further comprises an alkaline phosphatase amplification system. In alternative preferred embodiments, the methods further comprise at least one capture antibody, while in still further embodiments, the methods further comprise at least one detection antibody wherein the detection antibody is directed against the antibody directed against the soluble, non-fibrillar oligomeric amyloid β protein assemblies. In still further embodiments, the detection antibody further comprises at least one conjugated enzyme selected from the group consisting of horseradish peroxidase, alkaline phosphatase, urease, glucoamylase and β-galactosidase. In still further preferred embodiments, the methods further comprise the step of quantitating the at least one soluble, non-fibrillar oligomeric amyloid β protein assembly in the sample.

The present invention also provides kits for the detection of at least one soluble, non-fibrillar oligomeric amyloid β protein assembly comprising at least one antibody directed against at least one soluble, non-fibrillar oligomeric amyloid β protein assembly. In some embodiments, the kit comprises an immobilized support. In some preferred embodiments, the kit comprises an enzyme-linked immunosorbent assay kit. In still further embodiments, the kit further comprises components selected from the group consisting of needles, sample collection tubes, 96-well microtiter plates, instructions, at least one soluble, non-fibrillar oligomeric amyloid β protein assembly, an antibody-enzyme conjugate directed against a soluble, non-fibrillar oligomeric amyloid β protein assembly, at least one capture antibody, 96-well microtiter plates precoated with the at least one capture antibody, at least one coating buffer, at least one blocking buffer, distilled water, at least one enzyme-linked immunosorbent assay enzyme reaction substrate solution, and at least one amplifier system. In some preferred embodiments, the amplifier system is an alkaline phosphatase enzyme-linked immunosorbent assay amplifier system. The kits of the present invention may also contain any other useful components, including other antibodies (e.g., for detection of multiple different proteins) or other diagnostic reagents, therapeutic agents, instructions, education materials, and the like.

The present invention also provides methods for detecting at least one antibody directed against a soluble, non-fibrillar oligomeric amyloid β protein assembly, comprising: a) providing a sample suspected of containing at least one antibody directed against a soluble, non-fibrillar oligomeric amyloid β protein assembly and a detection antibody; b) contacting the sample with the soluble, non-fibrillar oligomeric amyloid β protein assembly, under conditions such that the antibody directed against a soluble, non-fibrillar oligomeric amyloid β protein assembly specifically binds to the soluble, non-fibrillar oligomeric amyloid β protein assembly to form an antigen-antibody complex; c) contacting the antigen-antibody complex with the detection antibody, under conditions such that the detection antibody specifically binds to the complex; and d) detecting the specific binding of the detection antibody to the antigen-antibody complex. In some preferred embodiments, the sample is selected from the group of samples consisting of blood, serous fluid, plasma, serum, cerebrospinal fluid, hybridoma conditioned culture medium, ascites fluid, and polyclonal antiserum. In some particularly preferred embodiments, the sample is from a subject, while in other preferred embodiments, the subject is human. In alternative preferred embodiments, the subject is selected from the group consisting of subjects displaying pathology resulting from Alzheimer's disease, subjects suspected of displaying pathology resulting from Alzheimer's disease, and subjects at risk of displaying pathology resulting from Alzheimer's disease. In still further preferred embodiments, the methods further comprise diagnosing Alzheimer's disease in the subject. In some preferred embodiments, the Alzheimer's disease is selected from the group consisting of late onset Alzheimer's disease, early onset Alzheimer's disease, familial Alzheimer's disease, and sporadic Alzheimer's disease. In preferred embodiments, the method comprises an enzyme-linked immunosorbent assay. In some preferred embodiments, the enzyme-linked immunosorbent assay is selected from the group consisting of direct enzyme-linked immunosorbent assays, indirect enzyme-linked immunosorbent assays, direct sandwich enzyme-linked immunosorbent assays, indirect sandwich enzyme-linked immunosorbent assays, and competitive enzyme-linked immunosorbent assays. In still further embodiments, the detection antibody further comprises a conjugated enzyme, wherein the conjugated enzyme is selected from the group of enzymes consisting of horseradish peroxidases, alkaline phosphatases, ureases, glucoamylases, and β-galactosidases. In additional embodiments, the enzyme-linked immunosorbent assay further comprises an alkaline phosphatase amplification system.

The present invention also provides kits for the detection of at least one antibody directed against at least one soluble, non-fibrillar oligomeric amyloid β protein assembly, comprising at least one soluble, non-fibrillar oligomeric amyloid β protein assembly and at least one detection antibody. In some embodiments, the kit comprises an immobilized support. In some preferred embodiments, the kit is an enzyme-linked immunosorbent assay kit. In some preferred embodiments, the kit comprises components selected from the group consisting of needles, sample collection tubes, 96-well microtiter plates, instructions, at least one purified antibody directed against at least one soluble, non-fibrillar oligomeric amyloid β protein assembly, at least one 96-well microtiter plate precoated with at least one soluble, non-fibrillar oligomeric amyloid β protein assembly, at least one coating buffer, at least one blocking buffer, distilled water, at least one enzyme reaction substrate solution, and at least one amplifier system. In some particularly preferred embodiments, the amplifier system is an alkaline phosphatase enzyme-linked immunosorbent assay amplifier system.

The present invention further provides methods for detecting at least one fibrillar amyloid β protein assembly, comprising the steps of: providing a sample suspected of containing at least one fibrillar amyloid β protein assembly and a monoclonal antibody that identifies fibrillar amyloid β protein assemblies, while not reacting with soluble, non-fibrillar oligomeric amyloid β protein assemblies; contacting the sample with the antibody under conditions such that the antibody binds to the fibrillar amyloid β protein assembly to form an antigen-antibody complex; and detecting the presence of the antigen-antibody complex. In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins. In some embodiments, the sample is selected from the group consisting of blood, plasma, serum, serous fluid, and cerebrospinal fluid. In some preferred embodiments, the sample is from a subject. In particularly preferred embodiments, the subject is a human. In further embodiments, the subject is selected from the group consisting of subjects displaying pathology resulting from Alzheimer's disease, subjects suspected of displaying pathology resulting from Alzheimer's disease, and subjects at risk of displaying pathology resulting from Alzheimer's disease. In some particularly preferred embodiments, the methods further comprise the step of diagnosing Alzheimer's disease. In additional particularly preferred embodiments, the Alzheimer's disease diagnosed using the methods of the present invention is selected from the group consisting of late onset Alzheimer's disease, early onset Alzheimer's disease, familial Alzheimer's disease and sporadic Alzheimer's disease. In some preferred embodiments, the methods further comprise the step of monitoring the efficacy of treatment of Alzheimer's disease.

In some preferred embodiments, the methods comprises an enzyme-linked immunosorbent assay. In particularly preferred embodiments, the enzyme-linked immunosorbent assay is selected from the group consisting of direct enzyme-linked immunosorbent assays, indirect enzyme-linked immunosorbent assays, direct sandwich enzyme-linked immunosorbent assays, indirect sandwich enzyme-linked immunosorbent assays, and competitive enzyme-linked immunosorbent assays. In alternative preferred embodiments, the antibody used in the methods of the present invention further comprises a conjugated enzyme, wherein the conjugated enzyme is selected from the group of enzymes consisting of horseradish peroxidases, alkaline phosphatases, ureases, glucoamylases, and β-galactosidases. In some particularly preferred embodiments, the enzyme-linked immunosorbent assay further comprises an alkaline phosphatase amplification system. In alternative preferred embodiments, the methods further comprise at least one capture antibody, while in still further embodiments, the methods further comprise at least one detection antibody wherein the detection antibody is directed against the antibody directed against the fibrillar amyloid β protein assemblies. In still further embodiments, the detection antibody further comprises at least one conjugated enzyme selected from the group consisting of horseradish peroxidase, alkaline phosphatase, urease, glucoamylase and β-galactosidase. In still further preferred embodiments, the methods further comprise the step of quantitating the at least fibrillar amyloid β protein assembly in the sample.

The present invention also provides kits for the detection of at least one fibrillar amyloid β protein assembly comprising at least one antibody directed against at least one fibrillar amyloid β protein assembly. In some embodiments, the kit comprises an immobilized support. In some preferred embodiments, the kit comprises an enzyme-linked immunosorbent assay kit. In still further embodiments, the kit further comprises components selected from the group consisting of needles, sample collection tubes, 96-well microtiter plates, instructions, an antibody-enzyme conjugate directed against a fibrillar amyloid β protein assembly, at least one capture antibody, 96-well microtiter plates precoated with the at least one capture antibody, at least one coating buffer, at least one blocking buffer, distilled water, at least one enzyme-linked immunosorbent assay enzyme reaction substrate solution, and at least one amplifier system. In some preferred embodiments, the amplifier system is an alkaline phosphatase enzyme-linked immunosorbent assay amplifier system.

### DESCRIPTION OF THE DRAWINGS

FIG. 1 depicts hybridoma screening by antigen/antibody blotting.

FIG. 2 shows an ELISA assay utilizing the monoclonal antibodies 6C3 and 7A2 (labeled “MOAB-2” and MOAB-1” respectively).

FIG. 3 shows Western blot analysis of unaggregated, oligomeric, and fibrillar preparations of amyloid β proteins using the monoclonal antibodies 6C3, 6E10, and 7A2.

FIG. 4 shows DAB staining and 10× light microscopy with monoclonal antibodies 6C3 and 7A2 in an AD brain.

FIG. 5 shows laser scanning confocal microscopy of AD brain slices using monoclonal antibody 6C3 and polyclonal antibody R1280.

FIG. 6 shows additional Western blot analysis of unaggregated, oligomeric, and fibrillar preparations of amyloid β proteins using the monoclonal antibodies 6C3, 6E10 and 7A2. Note: “MOAB-1” and “MOAB-2” correspond to 7A2 and 6C3 antibodies, respectively.

FIG. 7 shows dot blot analysis of unaggregated, oligomeric, and fibrillar preparations of amyloid β proteins using the monoclonal antibodies 6C3, 6E10 and 7A2. Note: “MOAB-1” and “MOAB-2” correspond to 7A2 and 6C3 antibodies, respectively.

### DEFINITIONS

To facilitate an understanding of the present invention, a number of terms and phrases are defined below:

As used herein, the terms “peptide,” “polypeptide” and “protein” all refer to a primary sequence of amino acids that are joined by covalent “peptide linkages.” In general, a peptide consists of a few amino acids, typically from 2-50 amino acids, and is shorter than a protein. The term “polypeptide” encompasses peptides and proteins. In some embodiments, the peptide, polypeptide or protein is synthetic, while in other embodiments, the peptide, polypeptide or protein are recombinant or naturally occurring. A synthetic peptide is a peptide that is produced by artificial means in vitro (i.e., was not produced in vivo).

The terms “sample” and “specimen” are used in their broadest sense and encompass samples or specimens obtained from any source. As used herein, the term “sample” is used to refer to biological samples obtained from animals (including humans), and encompasses fluids, solids, tissues, and gases. In preferred embodiments of this invention, biological samples include cerebrospinal fluid (CSF), serous fluid, urine, saliva, blood, and blood products such as plasma, serum and the like. However, these examples are not to be construed as limiting the types of samples that find use with the present invention.

As used herein, the terms “soluble, non-fibrillar oligomeric amyloid β protein assembly,” “oligomeric amyloid β protein assembly” and “oligomeric assembly” all refer to a protein assembly comprised of amyloid β proteins or peptides proteolytically derived from the transmembrane amyloid precursor protein (APP).

As used herein, the terms “fibrillar amyloid β protein assembly” and “fibrillar assembly” refers to a protein assembly comprised of amyloid β proteins or peptides proteolytically derived from the transmembrane amyloid precursor protein (APP).

As used herein, the term “oxidative stress” refers to the cytotoxic effects of oxygen radicals (i.e., superoxide anion, hydroxy radical, and hydrogen peroxide), generated as byproducts of metabolic processes that utilize molecular oxygen (See e.g., Coyle et al., Science 262:689-695 [1993]).

As used herein, the terms “host,” “subject” and “patient” refer to any animal, including but not limited to, human and non-human animals (e.g. rodents, arthropods, insects [e.g., Diptera], fish [e.g., zebrafish], non-human primates, ovines, bovines, ruminants, lagomorphs, porcines, caprines, equines, canines, felines, aves, etc.), that is studied, analyzed, tested, diagnosed or treated. As used herein, the terms “host,” “subject” and “patient” are used interchangeably.

As used herein, the terms “Alzheimer's disease” and “AD” refer to a neurodegenerative disorder and encompasses familial Alzheimer's disease and sporadic Alzheimer's disease. The term “familial Alzheimer's disease” refers to Alzheimer's disease associated with genetic factors (i.e., demonstrates inheritance) while “sporadic Alzheimer's disease” refers to Alzheimer's disease that is not associated with prior family history of the disease. Symptoms indicative of Alzheimer's disease in human subjects typically include, but are not limited to, mild to severe dementia, progressive impairment of memory (ranging from mild forgetfulness to disorientation and severe memory loss), poor visuo-spatial skills, personality changes, poor impulse control, poor judgement, distrust of others, increased stubbornness, restlessness, poor planning ability, poor decision making, and social withdrawal. In severe cases, patients lose the ability to use language and communicate, and require assistance in personal hygiene, eating and dressing, and are eventually bedridden. Hallmark pathologies within brain tissue include extracellular neuritic β-amyloid plaques, neurofibrillary tangles, neurofibrillary degeneration, granulovascular neuronal degeneration, synaptic loss, and extensive neuronal cell death.

As used herein, the term “early-onset Alzheimer's disease” refers to the classification used in Alzheimer's disease cases diagnosed as occurring before the age of 65. As used herein, the term “late-onset Alzheimer's disease” refers to the classification used in Alzheimer's disease cases diagnosed as occurring after the age of 65.

As used herein, the terms “subject having Alzheimer's disease” or “subject displaying symptoms or pathology indicative of Alzheimer's disease” “subjects suspected of displaying symptoms or pathology indicative of Alzheimer's disease” refer to a subject that is identified as having or likely to have Alzheimer's disease based on known Alzheimer's symptoms and pathology.

As used herein, the term “subject at risk of displaying pathology indicative of Alzheimer's disease” refers to a subject identified as being at risk for developing Alzheimer's disease (e.g., because of a familial inheritance pattern of Alzheimer's disease in the subject's family).

As used herein, the term “lesion” refers to a wound or injury, or to a pathologic change in a tissue. For example, the amyloid plaque lesions observed in the brains of patients having Alzheimer's disease are considered the hallmark pathology characteristic of the disease.

As used herein, the term “antibody” (or “antibodies”) refers to any immunoglobulin that binds specifically to an antigenic determinant, and specifically, binds to proteins identical or structurally related to the antigenic determinant that stimulated their production. Thus, antibodies are useful in assays to detect the antigen that stimulated their production. Monoclonal antibodies are derived from a single clone of B lymphocytes (i.e., B cells), and are generally homogeneous in structure and antigen specificity. Polyclonal antibodies originate from many different clones of antibody-producing cells, and thus are heterogenous in their structure and epitope specificity, but all recognize the same antigen. In some embodiments, monoclonal and polyclonal antibodies are used as crude preparations, while in preferred embodiments, these antibodies are purified. For example, in some embodiments, polyclonal antibodies contained in crude antiserum are used. Also, it is intended that the term “antibody” encompass any immunoglobulin (e.g., IgG, IgM, IgA, IgE, IgD, etc.) obtained from any source (e.g., humans, rodents, non-human primates, lagomorphs, caprines, bovines, equines, ovines, etc.).

As used herein, the terms “auto-antibody” or “auto-antibodies” refer to any immunoglobulin that binds specifically to an antigen that is native to the host organism that produced the antibody (i.e., the antigen is directed against “self” antigens). The presence of auto-antibodies is referred to herein as “autoimmunity.”

As used herein, the term “antigen” is used in reference to any substance that is capable of being recognized by an antibody. It is intended that this term encompass any antigen and “immunogen” (i.e., a substance that induces the formation of antibodies). Thus, in an immunogenic reaction, antibodies are produced in response to the presence of an antigen or portion of an antigen. The terms “antigen” and “immunogen” are used to refer to an individual macromolecule or to a homogeneous or heterogeneous population of antigenic macromolecules. It is intended that the terms antigen and immunogen encompass protein molecules or portions of protein molecules, that contains one or more epitopes. In many cases, antigens are also immunogens, thus the term “antigen” is often used interchangeably with the term “immunogen.” In some preferred embodiments, immunogenic substances are used as antigens in assays to detect the presence of appropriate antibodies in the serum of an immunized animal.

As used herein, the terms “antigen fragment” and “portion of an antigen” and the like are used in reference to a portion of an antigen. Antigen fragments or portions typically range in size, from a small percentage of the entire antigen to a large percentage, but not 100%, of the antigen. However, in situations where “at least a portion” of an antigen is specified, it is contemplated that the entire antigen is also present (i.e., it is not intended that the sample tested contain only a portion of an antigen). In some embodiments, antigen fragments and/or portions therof, comprise an “epitope” recognized by an antibody, while in other embodiments these fragments and/or portions do not comprise an epitope recognized by an antibody. In addition, in some embodiments, antigen fragments and/or portions are not immunogenic, while in preferred embodiments, the antigen fragments and/or portions are immunogenic.

The terms “antigenic determinant” and “epitope” as used herein refer to that portion of an antigen that makes contact with a particular antibody variable region. When a protein or fragment (or portion) of a protein is used to immunize a host animal, numerous regions of the protein are likely to induce the production of antibodies that bind specifically to a given region or three-dimensional structure on the protein (these regions and/or structures are referred to as “antigenic determinants”). In some settings, antigenic determinants compete with the intact antigen (i.e., the “immunogen” used to elicit the immune response) for binding to an antibody.

The terms “specific binding” and “specifically binding” when used in reference to the interaction between an antibody and an antigen describe an interaction that is dependent upon the presence of a particular structure (i.e., the antigenic determinant or epitope) on the antigen. In other words, the antibody recognizes and binds to a protein structure unique to the antigen, rather than binding to all proteins in general (i.e., non-specific binding).

As used herein the term “immunogenically-effective amount” refers to that amount of an immunogen required to invoke the production of protective levels of antibodies in a host upon vaccination.

As used herein, the term “adjuvant” is defined as a substance that enhances the immunogenicity of a coadministered antigen. If adjuvant is used, it is not intended that the present invention be limited to any particular type of adjuvant—or that the same adjuvant, once used, be used for all subsequent immunizations. The present invention contemplates many adjuvants, including but not limited to, keyhole limpet hemocyanin (KLH), agar beads, aluminum hydroxide or phosphate (alum), Freund's adjuvant (incomplete or complete), Quil A adjuvant and Gerbu adjuvant (Accurate Chemical and Scientific Corporation), and bacterins (i.e., killed preparations of bacterial cells, especially mycoplasma).

As used herein, the terms “purified” and “to purify” and “purification” refers to the removal or reduction of at least one contaminant from a sample. For example, antibodies are purified by removal of contaminating non-immunoglobulin proteins. Antibodies are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample (i.e., “enrichment” of an antibody).

As used herein, the term “immunoassay” refers to any assay that uses at least one specific antibody for the detection or quantitation of an antigen. Immunoassays include, but are not limited to, Western blots, ELISAs, radio-immunoassays, and immunofluorescence assays. Furthermore, many different ELISA formats are known to those in the art, any of which will find use in the present invention. However, it is not intended that the present invention be limited to these assays. In additional embodiments, other antigen-antibody reactions are used in the present invention, including but not limited to “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen), “complement fixation” (i.e., the use of complement in an antibody-antigen reaction method), and other methods commonly used in serology, immunology, immunocytochemistry, immunohistochemistry, and related fields.

The terms “Western blot,” “Western immunoblot” “immunoblot” and “Western” refer to the immunological analysis of protein(s), polypeptides or peptides that have been immobilized onto a membrane support. The proteins are first resolved by polyacrylamide gel electrophoresis (i.e., SDS-PAGE) to separate the proteins, followed by transfer of the protein from the gel to a solid support, such as nitrocellulose or a nylon membrane. The immobilized proteins are then exposed to an antibody having reactivity towards an antigen of interest. The binding of the antibody (i.e., the primary antibody) is detected by use of a secondary antibody that specifically binds the primary antibody. The secondary antibody is typically conjugated to an enzyme that permits visualization of the antigen-antibody complex by the production of a colored reaction product or catalyzes a luminescent enzymatic reaction (e.g., the ECL reagent, Amersham).

As used herein, the term “ELISA” refers to enzyme-linked immunosorbent assay (or EIA). Numerous ELISA methods and applications are known in the art, and are described in many references (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). In addition, there are numerous commercially available ELISA test systems.

One of the ELISA methods used in the present invention is a “direct ELISA,” where an antigen (e.g., an oligomeric or a fibrillar amyloid β protein assembly) in a sample is detected. In one embodiment of the direct ELISA, a sample containing antigen is exposed to a solid (i.e., stationary or immobilized) support (e.g., a microtiter plate well). The antigen within the sample becomes immobilized to the stationary phase, and is detected directly using an enzyme-conjugated antibody specific for the antigen.

In an alternative embodiment, an antibody specific for an antigen is detected in a sample. In this embodiment, a sample containing an antibody (e.g., an anti-oligomeric or an anti-fibrillar assembly antibody) is immobilized to a solid support (e.g., a microtiter plate well). The antigen-specific antibody is subsequently detected using purified antigen and an enzyme-conjugated antibody specific for the antigen.

In an alternative embodiment, an “indirect ELISA” is used. In one embodiment, an antigen (or antibody) is immobilized to a solid support (e.g., a microtiter plate well) as in the direct ELISA, but is detected indirectly by first adding an antigen-specific antibody (or antigen), then followed by the addition of a detection antibody specific for the antibody that specifically binds the antigen, also known as “species-specific” antibodies (e.g., a goat anti-rabbit antibody), that are available from various manufacturers known to those in the art (e.g., Santa Cruz Biotechnology; Zymed; and Pharmingen/Transduction Laboratories).

In other embodiments, a “sandwich ELISA” is used, where the antigen is immobilized on a solid support (e.g., a microtiter plate) via an antibody (i.e., a capture antibody) that is immobilized on the solid support and is able to bind the antigen of interest. Following the affixing of a suitable capture antibody to the immobilized phase, a sample is then added to the microtiter plate well, followed by washing. If the antigen of interest is present in the sample, it is bound to the capture antibody present on the support. In some embodiments, a sandwich ELISA is a “direct sandwich” ELISA, where the captured antigen is detected directly by using an enzyme-conjugated antibody directed against the antigen. Alternatively, in other embodiments, a sandwich ELISA is an “indirect sandwich” ELISA, where the captured antigen is detected indirectly by using an antibody directed against the antigen, that is then detected by another enzyme-conjugated antibody that binds the antigen-specific antibody, thus forming an antibody-antigen-antibody-anti-body complex. Suitable reporter reagents are then added to detect the third antibody. Alternatively, in some embodiments, any number of additional antibodies are added as necessary, in order to detect the antigen-antibody complex. In some preferred embodiments, these additional antibodies are labelled or tagged, so as to permit their visualization and/or quantitation.

As used herein, the term “capture antibody” refers to an antibody that is used in a sandwich ELISA to bind (i.e., capture) an antigen in a sample prior to detection of the antigen. For example, in some embodiments, the monoclonal anti-oligomeric or anti-fibrillar assembly antibodies of the present invention serve as a capture antibody when immobilized in a microtiter plate well. This capture antibody binds oligomeric or fibrillar amyloid β protein assembly antigens present in a sample added to the well. In one embodiment of the present invention, biotinylated capture antibodies are used in the present invention in conjunction with avidin-coated solid support. Another antibody (i.e., the detection antibody) is then used to bind and detect the antigen-antibody complex, in effect forming a “sandwich” comprised of antibody-antigen-antibody (i.e., a sandwich ELISA).

As used herein, a “detection antibody” is an antibody that carries a means for visualization or quantitation, that is typically a conjugated enzyme moiety that typically yields a colored or fluorescent reaction product following the addition of a suitable substrate. Conjugated enzymes commonly used with detection antibodies in the ELISA include horseradish peroxidase, urease, alkaline phosphatase, glucoamylase and β-galactosidase. In some embodiments, the detection antibody is directed against the antigen of interest, while in other embodiments, the detection antibody is not directed against the antigen of interest. In some embodiments, the detection antibody is an antibody directed against an antibody directed against the antigen of interest. Alternatively, the detection antibody is prepared with a label such as biotin, a fluorescent marker, or a radioisotope, and is detected and/or quantitated using this label.

As used herein, the terms “reporter reagent,” “reporter molecule,” “detection substrate” and “detection reagent” are used in reference to reagents that permit the detection and/or quantitation of an antibody bound to an antigen. For example, in some embodiments, the reporter reagent is a calorimetric substrate for an enzyme that has been conjugated to an antibody. Addition of a suitable substrate to the antibody-enzyme conjugate results in the production of a colorimetric or fluorimetric signal (e.g., following the binding of the conjugated antibody to the antigen of interest). Other reporter reagents include, but are not limited to, radioactive compounds. This definition also encompasses the use of biotin and avidin-based compounds (e.g., including but not limited to neutravidin and streptavidin) as part of the detection system.

As used herein, the term “signal” is used generally in reference to any detectable process that indicates that a reaction has occurred, for example, binding of antibody to antigen. It is contemplated that signals in the form of radioactivity, fluorimetric or colorimetric products/reagents will all find use with the present invention. In various embodiments of the present invention, the signal is assessed qualitatively, while in alternative embodiments, the signal is assessed quantitatively.

As used herein, the term “amplifier” is used in reference to a system that enhances the signal in a detection method, such as an ELISA (e.g., an alkaline phosphatase amplifier system used in an ELISA).

As used herein, the term “solid support” is used in reference to any solid or stationary material to which reagents such as antibodies, antigens, and other test components are attached. For example, in the ELISA method, the wells of microtiter plates provide solid supports. Other examples of solid supports include microscope slides, coverslips, beads, particles, cell culture flasks, gels, as well as many other suitable items.

As used herein, the term “kit” is used in reference to a combination of reagents and other materials that facilitate sample analysis. In some embodiments, the immunoassay kit of the present invention includes a suitable capture antibody, reporter antibody, antigen, detection reagents and amplifier system. Furthermore, in other embodiments, the kit includes, but is not limited to, components such as apparatus for sample collection, sample tubes, holders, trays, racks, dishes, plates, instructions to the kit user (including, for example, instructions and label as required by regulatory agencies), solutions or other chemical reagents, and samples to be used for standardization, normalization, and/or control samples.

As used herein, the term “in vitro” refers to an artificial environment and to processes or reactions that occur within an artificial environment. In vitro environments consist of, but are not limited to, controlled laboratory conditions. The term “in vivo” refers to the natural environment (e.g., an animal or a cell) and to processes or reactions that occur within that natural environment.

### DETAILED DESCRIPTION OF THE INVENTION

The cellular processes that underlie the cognitive decline and amnestic dementia associated with AD remain poorly understood. Amyloid β protein is a peptide that is proteolytically derived from the transmembrane amyloid precursor protein (APP). Evidence from numerous studies supports the hypothesis that amyloid β protein accumulation is causally linked to AD. However a causal linkage between pathology, in terms of senile plaques composed primarily of deposited fibrillar amyloid β protein, and symptomology in terms of cognitive impairment and dementia, has not been forthcoming. An emerging hypothesis that reconciles this apparent disconnect focuses on small soluble assemblies of amyloid β protein.

Recent experimental evidence has demonstrated that these oligomeric conformations are directly involved in many of the destructive processes that result in neurodegeneration. Oligomeric amyloid β protein assemblies have been isolated from brain, plasma and CSF and soluble amyloid β protein concentrations in brain are correlated with the severity of AD. Furthermore, autosomal dominant mutations in the amyloid precursor protein (APP) and the presenilins (PS) increase the amount of amyloid β1-42, a proteolytic product of APP, and always result in AD. This provides powerful genetic evidence that some form of the amyloid β protein is involved in the disease process.

The role of amyloid β protein in the AD disease process has been put forth as the comprehensive “amyloid hypothesis” (Selkoe, J. Neuorpath. Exp. Neurol., 53: 438 [1991]). In it, it is stated that the production and deposition of amyloid β protein fibrils in plaques induces a neurotoxic event. Presumably, this initial event culminates in the intracellular accumulation of tau polymers as neurofibrillary tangles leading to neuronal dysfunction and death. The amyloid hypothesis gained wide acceptance when initial reports indicated that fibrillar amyloid β protein was cytotoxic in vitro (Pike et al., J. Neuroscience, 13: 1676 [1993]; Schenk et al., J. Med. Chem., 38: 4141 [1995]). However, the veracity of the amyloid hypothesis is challenged by a seeming disconnect between aspect of the plaque pathology and AD symptomatology.

In one of the first discoveries challenging this hypothesis, pathologists noted that the correlation between the number, location and distribution of senile plaques (amyloid load) and the degree of dementia as assessed neuropsychologically was poor at best (Swaab et al., Connections, Cognition and Alzheimer's Disease, Springer-Verlag, Berlin/NY [1997]. Second, amyloid deposition in senile plaques is temporarily dissociated from cognitive defects in transgenic mouse models overexpressing APP and PS (Hsai et al., Proc. Natl. Acad. Sci., 96: 3228 [1999]; Holcomb et al., Nature Med., 4: 97 [1998]; Chui et al., Nature Med., 5: 560 [1999]; Moeehars et al., J. Biol. Chem., 274: 6483 [1999]). Finally, several therapeutics designed to block fibril formation have been unsuccessful in delaying AD symptoms (Schenk et al., J. Med. Chem., 38: 4141 [1995]; Soto, Mol. Med. Today, 5: 343 [1999]).

On the other hand, several lines of evidence suggest that soluble oligomeric amyloid β protein species, as distinct from large, fibrillar aggregates, do correlate with AD pathology. The concentration of soluble amyloid β protein in the brain is highly correlated with disease severity (Lue et al., Amer. J. Path., 155: 853 [1999]; McLean et al., Ann Neurol., 46: 860 [1999]). Furthermore, results from in vitro experiments demonstrate that soluble oligomeric amyloid β proteins not only can readily form, but that these species are highly cytotoxic (Roher et al., J. Biol. Chem., 271: 20631 [1996]; Hartley et al., J. Neurosci., 19: 8876 [1999]; Lambert et al., Proc. Natl. Acad. Sci., 95: 6448 [1998]; Oda et al., Exp. Neurol., 136: 22 [1995]). For example, the process of amyloid β protein oligomerization is enhanced in the media of cells expressing the APP or PS mutations, providing a possible connection between toxic oligomer formation and AD genetics (Podlinsy et al., Biochemistry, 37: 3602 [1998]).

While a compelling argument can be made for the relevance of a toxic, diffusible amyloid β protein oligomer, the presence of this amyloid β protein species has not been demonstrated in normal or AD brain by immunohistochemistry. This is primarily due to a lack of antibodies that can distinguish different conformational forms of the amyloid β protein. Hence, one of the primary limitations to properly dissecting the role of fibrillar versus oligomeric amyloid β protein assemblies has been the lack of conformational-specific antibodies that can distinguish between these two aggregates of the amyloid β protein.

Hence, although it is not clear whether amyloid β protein accumulation causes Alzheimer's disease or is an effect of Alzheimer's disease, considerable evidence has strengthened the view that amyloid β protein accumulation is the causative agent of Alzheimer's disease. However, it is not necessary to understand the cause or effect of amyloid β protein accumulation in Alzheimer's disease in order to practice the present invention, nor is it intended that the present invention be limited to any particular mechanism or mechanisms of disease genesis or toxicity. Indeed, an understanding of any of the mechanisms of pathogenesis are not necessary in order to use the present invention.

In some embodiments, the present invention provides monoclonal antibodies that specifically bind to soluble, non-fibrillar oligomeric amyloid β protein assemblies while not reacting with fibrillar amyloid β protein assemblies, and monoclonal antibodies that specifically bind to fibrillar amyloid β protein assemblies that do not react with soluble, non-fibrillar oligomeric amyloid β protein assemblies (e.g., as shown in Examples 2 and 3). In some embodiments, the soluble, non-fibrillar oligomeric amyloid β protein assemblies comprise 2-12 amyloid β proteins. In some embodiments, the fibrillar amyloid β protein assemblies comprise more than 12 amyloid β proteins. The present invention, however, is not limited by the number of amyloid β proteins present in the non-fibrillar oligomeric assemblies or fibrillar assemblies. In further embodiments, these antibodies are used to identify soluble, non-fibrillar oligomeric amyloid β protein assemblies or fibrillar amyloid β protein assemblies, respectively. However, it is not intended that the use of these antibodies be limited to identifying oligomeric and filbrillar forms of the amyloid β protein. For example, these antibodies may also be used to inhibit or to precipitate the assembly of amyloid β protein fibrils.

Additionally, the antibodies of the present invention find other uses, including enzyme-linked immunosorbent assays (ELISAs) (e.g., as shown in Example 3), Western blotting (e.g., as shown in Example 4), radioimmunoassays (RIAs), immunofluorescence assays (IFAs), immunoprecipitation, immunohistochemistry (e.g., as shown in Example 5), laser scanning confocal microscopy (e.g., as shown in Example 6) and clinical diagnostic applications using methods known in the art (See e.g., Harlow and Lane (eds.), Antibodies: A Laboratory Manual, Cold Spring Harbor Laboratory Press [1988]; Ausubel et al. (eds.), Current Protocols in Molecular Biology, Vol. 1-4, John Wiley & Sons, Inc., New York [1994]; and Laurino et al., Ann. Clin. Lab Sci., 29(3):158-166 [1999]).

It is not intended that the production of antibodies of the present invention be limited to any particular method. Indeed, it is contemplated that the antibodies be prepared by any suitable method. Numerous methods for the production and purification of monoclonal antibodies are well known in the art (See e.g., Sambrook et al. (eds.), Molecular Cloning, Cold Spring Harbor Laboratory Press [1989]; Harlow and Lane (eds.), Antibodies: A Laboratory Manual, Cold Spring Harbor Laboratory Press [1988]; Ausubel et al. (eds.), Current Protocols in Molecular Biology, p. 11.4.2-11.15.4, John Wiley & Sons, Inc., New York [1994]; Kohler and Milstein, Nature 256:495-497 [1975]; Kozbor et al., Immunol. Today 4:72 [1983]; and Cole et al., in Monoclonal Antibodies and Cancer Therapy, Alan R. Liss, Inc., pp. 77-96 [1985]).

In addition, in other embodiments, any suitable amyloid β protein or fragment thereof, is used as an immunogen. (e.g., generation of immunogens is described in Example 1). In some embodiments, the immunogen is native, while in other embodiments, the immunogen is synthetic (i.e., recombinant or produced by in vitro chemical synthesis). Similarly, it is not intended that the present invention be limited to any particular amyloid β protein-derived immunogen, immunization method, immunization schedule, animal species, test protocol for determining antibody production or antibody purification method.

In some embodiments, the monoclonal antibody preparation of the present invention is purified from crude antiserum, hybridoma or cell culture supernatant, ascites fluid, or other starting material using any conventional method. Such purification methods include, but are not limited to, protein A affinity, protein G affinity, ammonium sulfate precipitation, ion exchange chromatography, gel filtration, and immunoaffinity chromatography (See, e.g., Harlow and Lane (eds.), Antibodies: A Laboratory Manual, Cold Spring Harbor Laboratory Press [1988]; and Ausubel et al. (eds.), Current Protocols in Molecular Biology, Ch. 11, John Wiley & Sons, Inc., New York [1994]).

Clonal selection of hybridomas is performed by incubating supernatants from each clone in two ELISA wells, one with amyloid β protein oligomers attached and the other with fibrils attached (e.g., described in Example 2). Clonal supernatants from oligomer-immunized mice that are positive on the oligomer-attached plate but negative on the fibril-attached plate are selected for further subcloning. This dual selection protocol is repeated for screening fusion of splenocytes obtained from fibril-immunized mice.

The specificity of antibodies produced from hybridomas during the development of the present invention can be further characterized by antibody/antigen spotting and Western blotting (e.g., as described in Examples 2 and 3, respectively, below).

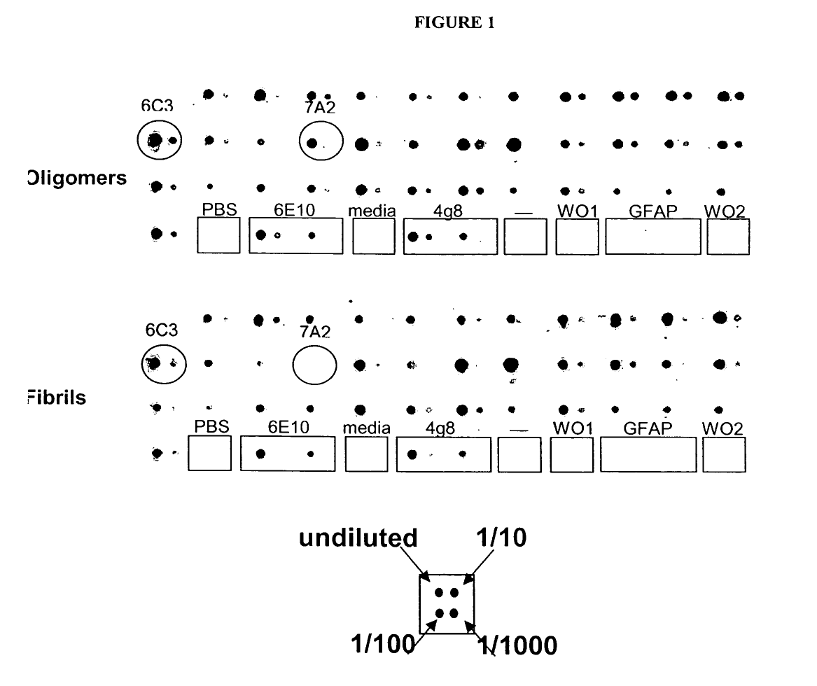

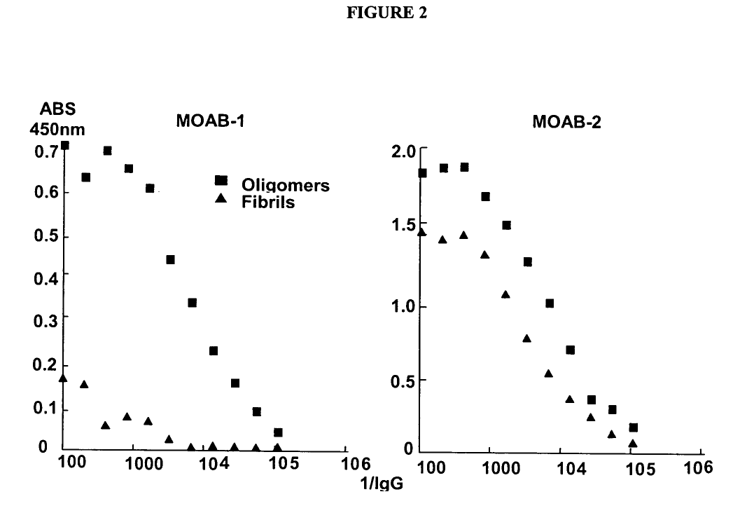

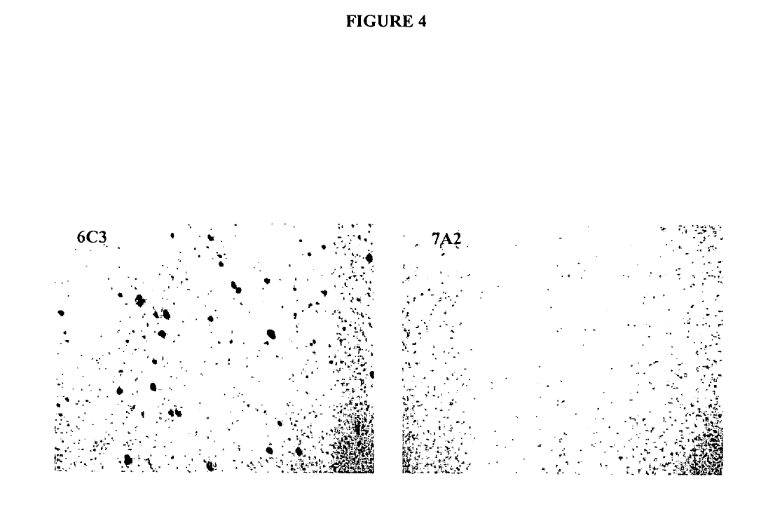

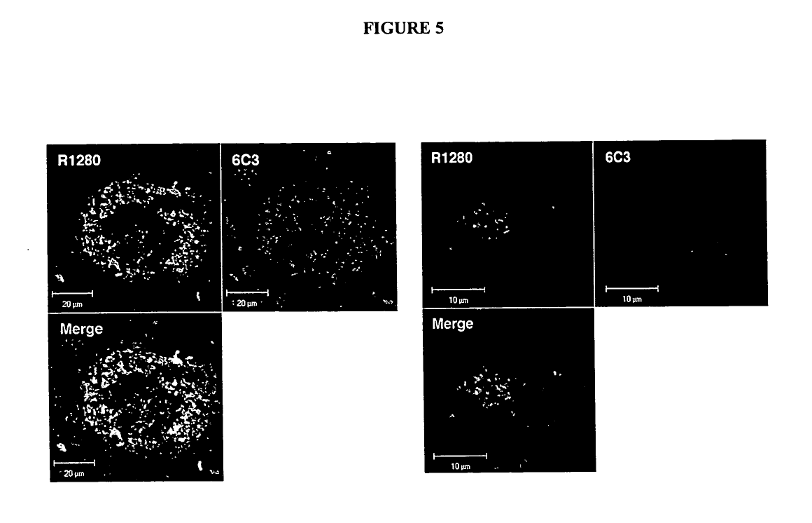

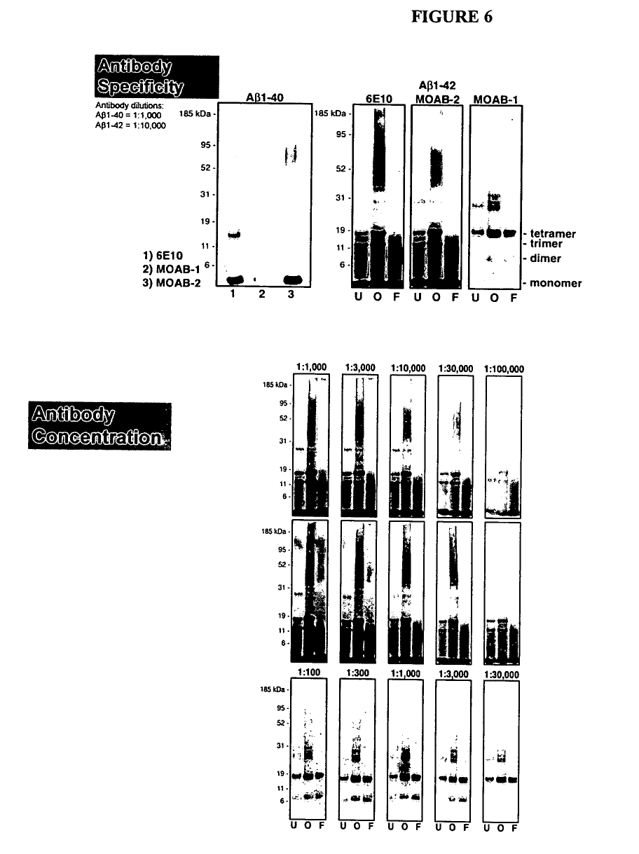

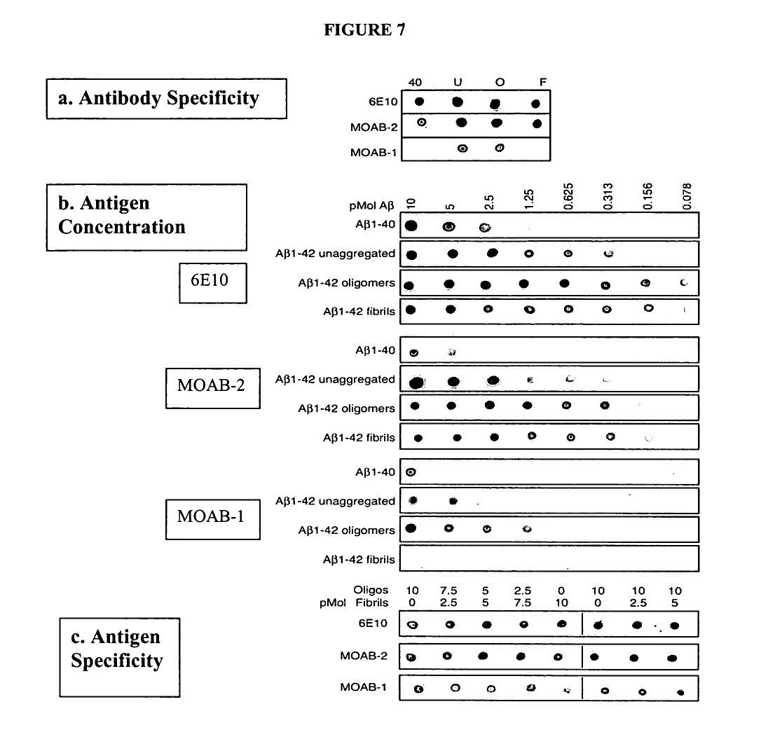

Experiments conducted during the course of development of the present invention showed an antigen/antibody screen yielding one oligomer-specific antibody. The oligomer-specific antibody (7A2) showed little recognition of fibrils by antigen/antibody blotting (e.g., see Example 2 and FIG. 1) and ELISA (e.g., see Example 3 and FIG. 2). By Western analysis, 7A2 detected oligomeric (primarily dimer, tetramer and larger oligomers between approximately 27 and 44 kDa) amyloid β protein assemblies (e.g., see Example 7, FIGS. 6 and 7), whereas 6E10 and 6C3 antibodies detected multiple forms of amyloid β protein1-42 including monomer, trimer, tetramer and oligomers between approximately 27 and 80 kDa. 7A2 oligomer specificity was retained over a wide range of antibody and antigen concentrations, and in the presence of increasing concentrations of fibrillar amyloid β protein1-42 (e.g., see Example 7, FIGS. 6 and 7). In sections from AD brain, little immunoreactivity of 7A2 antibody with fibrillar amyloid protein was detected (e.g., see Example 5 and FIG. 4). 6C3 antibody was used in bright field immunohistochemistry and laser scanning confocal microscopy (LSCM) to detect structures resembling diffuse amyloid plaques not detected by AD polyclonal antibodies in hippocampal sections from the brains of neuropsychologically well-characterized AD patients (e.g., see Example 6 and FIG. 5).

It is known that oligomeric amyloid β protein assemblies are present in human blood and cerebrospinal fluid (CSF) of living subjects. It is contemplated that oligomeric amyloid β protein assemblies are also present in the blood, serous fluid and/or CSF of living subjects. It is contemplated that the presence of oligomeric amyloid β protein assemblies, or the presence of oligomeric assemblies above a threshold level, in these fluids is diagnostic of Alzheimer's disease. Thus, the present invention provides methods and compositions for the diagnosis and prognosis of Alzheimer's disease. Indeed, the present invention provides compositions and methods to analyze disease severity, and the efficacy of Alzheimer's disease therapies. It is contemplated that subjects identified as having higher levels of oligomeric amyloid β protein assemblies (e.g., in blood, serous fluid or CSF) have more advanced Alzheimer's disease than subjects showing lower levels of oligomeric amyloid β protein assemblies. It is contemplated that by monitoring the levels of oligomeric amyloid β protein assemblies in blood, serous fluid and/or CSF of patients undergoing treatment for Alzheimer's disease, determinations regarding the effectiveness of treatment regimes are possible. For example, reduced levels of oligomeric amyloid β protein assemblies over time indicate that the treatment used to treat a subject with Alzheimer's disease is effective.

It is contemplated that the present invention will find use in testing subjects such as those who have been previously diagnosed with Alzheimer's disease, those who are suspected of having Alzheimer's disease, and those at risk of developing Alzheimer's disease. For example, patients diagnosed with dementia, in particular, those patients who were previously clinically normal, are suitable subjects. However, it is not intended that the present invention be limited to use with any particular subject or patient types. The methods of the present invention are also useful for detecting early onset Alzheimer's disease and late onset Alzheimer's disease, as well as for detecting sporadic Alzheimer's disease and familial Alzheimer's disease.

The present invention also provides compositions and methods for the detection and quantitation (i.e., measurement) of oligomeric and fibrillar amyloid β protein assemblies in the blood, serous fluid and CSF. Standard techniques known in the art are easily adapted to quantitate the levels of circulating oligomeric and fibrillar amyloid β protein assemblies in blood, serous fluid and/or CSF samples, including but not limited to, ELISA.

Factors contributing to the success of the ELISA methods of the present invention include their sensitivity, versatility, long reagent shelf-life, ease of preparation of reagents, non-radioactive reagents, and assay speed. Furthermore, in some embodiments, the assay is quantitative. In addition, reagents and equipment designed specifically for use in ELISA protocols are readily available from numerous manufacturers, including Pierce Chemical Company, Bio-Rad, Dynatech Industries, GibcoBRL/Life Technologies, Fisher Scientific, and Promega.

Many ELISA applications and formats have been described. Various sources provide discussion of ELISA chemistry, applications, and detailed protocols (See e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]; and Laurino et al., Ann. Clin. Lab Sci., 29(3):158-166 [1999]).

In preferred embodiments of the present invention, ELISA methods for quantitation of antigen are provided. In some of these methods, the antigen (e.g., oligomeric amyloid β protein) is first immobilized on a solid support (e.g., in a microtiter plate well). Detection and quantitation of the immobilized antigen is accomplished by use of an antibody-enzyme conjugate capable of binding to the immobilized antigen and producing a quantifiable signal. In some embodiments, the amount of antigen present is directly proportional to the amount of enzyme reaction product produced after the addition of an appropriate enzyme substrate.

As indicated previously, enzymes commonly used in ELISAs include horseradish peroxidase (HRPO), urease, alkaline phosphatase, glucoamylase and β-galactosidase. Protocols for the preparation of suitable antibody-enzyme conjugates are well known in the art. The present invention provides methods for the preparation of an antibody-enzyme (i.e., HRPO enzyme) conjugate that specifically recognizes the antigen of interest (i.e., oligomeric or fibrillar amyloid β protein assemblies) for use in immunoassay (e.g. ELISA) methods for detection of Alzheimer's disease. It is not intended that the present invention be limited to the antibody-enzyme conjugation method provided herein, as those of skill in the art will recognize other methods for antibody-enzyme conjugation that find use with the present invention.

Conjugation of enzymes to antibodies involves the formation of a stable, covalent linkage between an enzyme (e.g., HRPO or alkaline phosphatase) and the antibody (e.g., the monoclonal anti-oligomeric amyloid β protein assembly antibody or the monoclonal anti-fibrillar amyloid β protein assembly antibody), where neither the antigen-binding site of the antibody nor the active site of the enzyme is functionally altered.

The conjugation of antibody and HRPO is dependent on the generation of aldehyde groups by periodate oxidation of the carbohydrate moieties on HRPO (Nakane and Kawaoi, J. Histochem. Cytochem., 22:1084-1091 [1988]). Combination of these active aldehydes with amino groups on the antibody forms Schiff bases that, upon reduction by sodium borohydride, become stable.

Protocols to make antibody-enzyme conjugates using urease or alkaline phosphatase enzymes are also known in the art (Healey et al., Clin. Chim. Acta 134:51-58 [1983]; Voller et al., Bull. W. H. O., 53:55-65 [1976]; and Jeanson et al., J. Immunol. Methods 111:261-270 [1988]). For urease conjugation, cross-linking of the urease enzyme (e.g., Urease Type VII, Sigma No. U0376) and antibody using m-maleimidobenzoyl N-hydroxysuccinimide ester (MBS) is achieved through benzoylation of free amino groups on the antibody. This is followed by thiolation of the maleimide moiety of MBS by the cysteine sulfhydryl groups of urease. To prepare an alkaline phosphatase-antibody conjugate, a one-step glutaraldehyde method is the simplest procedure (Voller et al., Bull. W. H. O., 53:55-65 [1976]). This antibody-alkaline phosphatase conjugation protocol uses an enzyme immunoassay grade of the alkaline phosphatase enzyme.

The end product of an ELISA is a signal typically observed as the development of color or fluorescence. Typically, this signal is read (i.e., quantitated) using a suitable spectrocolorimeter (i.e., a spectrophotometer) or spectrofluorometer. The amount of color or fluorescence is directly proportional to the amount of immobilized antigen. In some embodiments of the present invention, the amount of antigen in a sample (e.g., the amount of oligomeric or fibrillar amyloid β protein assemblies in a blood or CSF sample) is quantitated by comparing results obtained for the sample with a series of control wells containing known concentrations of the antigen (i.e., a standard concentration curve). A negative control is also included in the assay system.

It is contemplated that any suitable chromogenic or fluorogenic substrates will find use with the enzyme-conjugated antibodies of the present invention. In some embodiments of the present invention, the substrate p-nitrophenyl phosphate (NPP) in diethanolamine is the preferred substrate for use in calorimetric ELISA methods, and 4-methylumbelliferyl phosphate (MUP) is the preferred alkaline phosphatase substrate in fluorometric ELISA methods.

The present invention provides various ELISA protocols for the detection and/or quantitation of oligomeric or fibrillar amyloid β protein assemblies in a sample. In one embodiment, the present invention provides a “direct ELISA” for the detection of oligomeric or fibrillar amyloid β protein assemblies in a sample. In some embodiments, the antigen of interest in a sample (i.e., the oligomeric or fibrillar amyloid β protein assembly) is bound (along with unrelated antigens) to the solid support (e.g., a microtiter plate well). The immobilized antigen is then directly detected by the antigen-specific enzyme-conjugated antibody, also provided by the present invention. Addition of an appropriate detection substrate results in color development or fluorescence that is proportional to the amount of antigen present in the well.

In another embodiment, the present invention provides an indirect ELISA for the detection of antigen in a sample. In this embodiment, antigen of interest in a sample is immobilized (along with unrelated antigens) to a solid support (e.g., a microtiter plate well) as in the direct ELISA, but is detected indirectly by first adding an antigen-specific antibody, then followed by the addition of a detection antibody specific for the antibody that specifically binds the antigen, also known as “species-specific” antibodies (e.g., a goat anti-rabbit antibody), which are available from various manufacturers known to one in the art (e.g., Santa Cruz Biotechnology; Zymed; and Pharmingen/Transduction Laboratories).

In some embodiments, the concentration of sample added to each well is titrated, so as to produce an antigen concentration curve. In other embodiments, the concentration of conjugated antibody is titrated. Indeed, such titrations are typically performed during the initial development of ELISA systems.

In another embodiment, the present invention provides “sandwich ELISA” methods, in which the antigen in a sample is immobilized on the solid support by a “capture antibody” that has been previously bound to the solid support. In general, the sandwich ELISA method is more sensitive than other configurations, and is capable of detecting 0.1-1.0 ng/ml protein antigen. As indicated above, the sandwich ELISA method involves pre-binding the “capture antibody” which recognizes the antigen of interest (i.e., the oligomeric or fibrillar amyloid β protein assemblies) to the solid support (e.g. wells of the microtiter plate). In some embodiments, a biotinylated capture antibody is used in conjunction with avidin-coated wells. Test samples and controls are then added to the wells containing the capture antibody. If antigen is present in the samples and/or controls, it is bound by the capture antibody.

In some embodiments, after a washing step, detection of antigen that has been immobilized by the capture antibody is detected directly (i.e., a direct sandwich ELISA). In other embodiments detection of antigen that has been immobilized by the capture antibody is detected indirectly (i.e., an indirect sandwich ELISA). In the direct sandwich ELISA, the captured antigen is detected using an antigen-specific enzyme-conjugated antibody. In the indirect sandwich ELISA, the captured antigen is detected by using an antibody directed against the antigen, which is then detected by another enzyme-conjugated antibody which binds the antigen-specific antibody, thus forming an antibody-antigen-antibody—antibody complex. In both the direct and indirect sandwich ELISAs, addition of a suitable detection substrate results in color development or fluorescence that is proportional to the amount of antigen that is present in the well.

In the sandwich ELISA, the capture antibody used is typically different from the second antibody (the “detection antibody”). The choice of the capture antibody is empirical, as some pairwise combinations of capture antibody and detection antibody are more or less effective than other combinations. The same monoclonal antibody must not be used as both the capture antibody and the conjugated detection antibody, since recognition of a single epitope by the capture antibody will preclude the enzyme-conjugated detection antibody from binding to the antigen. However, in some embodiments, two different monoclonal antibodies that recognize different epitopes are used in this assay.

Furthermore, it is not intended that the present invention be limited to the direct ELISA and sandwich ELISA protocols particularly described herein, as the art knows well numerous alternative ELISA protocols that also find use in the present invention (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; and Ausubel et al (eds.), Current Protocols in Molecular Biology, Ch. 11, John Wiley & Sons, Inc., New York [1994]). Thus, any suitable ELISA method including, but not limited to, competitive ELISAs also find use with the present invention.

In another embodiment, the present invention provides methods for the detection and quantitation of oligomeric or fibrillar amyloid β protein assembly reactive antibodies. Briefly, in some embodiments, variations of indirect ELISAs are used. In preferred embodiments, antigens (i.e., oligomeric or fibrillar amyloid β protein assemblies) are first used to coat the wells of a 96-well microtiter plate. The test sample is then added to the antigen-coated wells. If the test sample contains oligomeric or fibrillar amyloid β protein assembly reactive antibodies, these antibodies specifically bind to the purified antigen coating the well. The oligomeric or fibrillar amyloid β protein assembly reactive antibodies are then visualized by the addition of a second detection antibody, where the detection antibody is coupled to an enzyme and is species-specific or isotype-specific for anti-oligomeric or anti-fibrillar amyloid β protein assembly antibody. As with all ELISA methods, appropriate negative and positive controls are included in order to ensure the reliability of the assay results.

It is contemplated that patients with Alzheimer's disease produce oligomeric or fibrillar amyloid β protein assembly-reactive auto-antibodies, and an ELISA to detect oligomeric or fibrillar amyloid β protein assembly reactive antibodies in such samples will find use in the diagnosis of Alzheimer's disease. It is further contemplated that the presence of anti-oligomeric or anti-fibrillar amyloid β protein assembly auto-antibodies in a patient is diagnostic of Alzheimer's disease.

It is also contemplated that the present invention will find use in detection of oligomeric or fibrillar amyloid β protein assembly reactive antibodies in various other settings (e.g., in the screening of monoclonal hybridoma culture supernatants [i.e., conditioned hybridoma culture medium], ascites fluid and/or polyclonal antisera).

The present invention also provides ELISA amplification systems. These embodiments produce at least 10-fold, and more preferably, a 500-fold increase in sensitivity over traditional alkaline phosphatase-based ELISAs. In one preferred embodiment of the ELISA amplification protocol, bound alkaline phosphatase acts on an NADPH substrate, whose reaction product initiates a secondary enzymatic reaction resulting in a colored product. Each reaction product from the first reaction initiates many cycles of the second reaction in order to amplify the signal (See e.g., Bio-Rad ELISA Amplification System, Cat. No. 19589-019).

The present invention also provides ELISA kits for the detection of antibodies and/or antigen. In addition, in some embodiments, the kits are customized for various applications. However, it is not intended that the kits of the present invention be limited to any particular format or design. In some embodiments, the kits of the present invention include, but are not limited to, materials for sample collection (e.g., spinal and/or venipuncture needles), tubes (e.g., sample collection tubes and reagent tubes), holders, trays, racks, dishes, plates (e.g., 96-well microtiter plates), instructions to the kit user, solutions or other chemical reagents, and samples to be used for standardization, and/or normalization, as well as positive and negative controls. In particularly preferred embodiments, reagents included in ELISA kits specifically intended for the detection of oligomeric or fibrillar amyloid β protein assemblies or anti-oligomeric or anti-fibrillar amyloid β protein assembly antibodies include control oligomeric amyloid β protein assemblies, anti-oligomeric and/or anti-fibrillar amyloid β protein assembly antibodies, anti-oligomeric and/or anti-fibrillar amyloid β protein assembly antibody-enzyme conjugate, 96-well microtiter plates precoated with control RA-NDA peptide, suitable capture antibody, 96-well microtiter plates precoated with a suitable oligomeric and/or anti-fibrillar amyloid β protein assembly capture antibody, buffers (e.g., coating buffer, blocking buffer, and distilled water), enzyme reaction substrate and premixed enzyme substrate solutions.

It is contemplated that the compositions and methods of the present invention will find use in various settings, including research and clinical diagnostics. For example, the anti-oligomeric and/or anti-fibrillar amyloid β protein assembly antibodies of the present invention also find use in studies of APP metabolism and in in situ hybridization studies of brain tissue sections to observe Alzheimer's disease pathology. In addition, methods to quantitate oligomeric and/or fibrillar amyloid β protein assemblies in samples find use in monitoring and/or determining the effectiveness of Alzheimer's disease treatment, as it is contemplated that decreasing levels of oligomeric amyloid β protein assemblies in a subject's samples over time indicates the effectiveness of an Alzheimer's disease treatment. Uses of the compositions and methods provided by the present invention encompass human and non-human subjects and samples from those subjects, and also encompass research as well as diagnostic applications. Thus, it is not intended that the present invention be limited to any particular subject and/or application setting.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: degree. C. (degrees Centigrade); cm (centimeters); g (grams); 1 or L (liters); .mu.g (micrograms); .mu.l (microliters); .mu.m (micrometers); .mu.M (micromolar); lmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); pmol (picomoles); Aldrich (Sigma/Aldrich, Milwaukee, Wis.); Amersham (Amersham/Pharmacia Biotech, Piscataway, N.J.); Bio-Rad (Bio-Rad Laboratories, Hercules, Calif.), Boehringer Mannheim (Boehringer Mannheim Corporation, Indianapolis, Ind.); Dynex (Dynex Technologies, Inc., Chantilly, Va.); Fisher Scientific (Fisher Scientific, Pittsburgh, Pa.), GiboBRL/Life Technologies (GibcoBRL/Life Technologies, Gaithersburg, Md.), Oncogene Research Products (Oncogene Research Products, Cambridge, Mass.); Pharmingen/Transduction Laboratories (Pharmingen/Transduction Laboratories/Becton Dickinson Company, San Diego, Calif.); Pierce Chemical Company (Pierce Chemical Company, Rockford, Ill.); Promega (Promega Corporation, Madison, Wis.); Santa Cruz Biotechnology (Santa Cruz Biotechnology, Inc., Santa Cruz, Calif.); Sigma (Sigma Chemical Co., St. Louis, Mo.); and Zymed (Zymed Laboratories, Inc., South San Francisco, Calif.).

### Example 1

### Materials and Methods

Generation of fibrillar or oligomeric immunogens for generation of hybridomas. Pretreated stocks of amyloid β protein stored as HFIP films are monomerized in DMSO, then aggregated in dilute acid at low salt (10 mM HCL) to produce fibrillar amyloid β protein, or, in cell culture media (phenyl-free F12, Gibco BRL) containing physiologic salt and pH levels to produce oligomeric structures. The amyloid β protein fibrils produced under the acidic conditions have diameters that measure approximately ˜4 nm in z-height and extend for several microns. Oligomers produced in cell culture media range in size from ˜2 nm in z-height.

Immunizations. Female Balb/c mice are immunized with amyloid β protein oligomers or fibrils produced as described above. The immunogens employed are suspended in Freunds Incomplete Adjuvant at a concentration of 1 μg/μl. A total of 200 μg is injected subcutaneously every 2 weeks until the serum titer of the mouse is half-maximal at a dilution of 2×10⁻⁴ as judged by ELISA with 50 ng of amyloid β protein oligomers or fibrils attached per well in the solid phase.

Hybridoma production and clonal selection. Once the desired serum titer is attained, immune spleens are removed from the mice, dissociated, and fused with SP2/o myeloma cells. The resultant cell suspension is plated in 96 well plates, HAT selected and cultured for 10-14 days to allow clonal growth. Initial clonal selection is performed by incubating supernatants from each clone in two ELISA wells, one with amyloid β protein oligomers attached and the other with fibrils attached. Clonal supernatants from oligomer-immunized mice that are positive on the oligomer-attached plate but negative (or exhibit a two-fold or greater signal diminution) on the fibril-attached plate are selected for further subcloning. This dual selection protocol is repeated for screening fusion of splenocytes obtained from fibril-immunized mice. In this case, clones are selected that bind to the fibrils but not to the oligomers.

Subcloning and antibody production. Mother clones are subcloned 3-4 times to assure monoclonality and allow the hybrids to stabilize. Antibodies are isotyped and the stable clones are adapted to serum-free medium and placed in a bioreactor for antibody expression. Purified, homogenous monoclonal antibodies are then stored at 1 mg/ml in borate buffered saline containing 50% glycerol.

### Example 2

### Screening of Hybridoma Supernatants by Antigen/Antibody Blotting

In order to determine the specificity of the antibodies made by the hybridomas, supernatants of the hybridomas were screened by antigen/antibody blotting. 5 μM amyloid β protein1-42 oligomer or fibril solutions were incubated with Immobilon-P membranes at room temperature for 30 minutes. Following rinsing and blocking, hybridoma supernatant was spotted onto membrane with a 96-pin replicator.

This method identified several hybridomas making antibodies which appeared specific for oligomer amyloid β protein assemblies and not the fibrillar form (FIG. 1). Monoclonal antibodies from the hybridoma 7A2 are oligomer-specific and show little recognition of fibrils by antigen/antibody blotting. In contrast, antibodies from the hybridoma 6C3 do not demonstrate specificity for oligomers or fibrils by antigen/antibody blotting.

### Example 3

### ELISA Titer: Oligomer-Versus Fibril Specificity for 6C3 and 7A2

Antibodies from hybridomas 7A2 and 6C3 were purified to homogeneity and further characterized in an ELISA assay. Serial dilutions of the purified antibodies were incubated with 25 ng of fibrillar or oligomeric amyloid β protein assemblies in the solid phase. 7A2 antibodies do not recognize fibrils by antigen/antibody blotting (FIG. 1). Additionally, standard ELISA shows that 7A2 antibodies display significant affinity for oligomeric assemblies while not displaying a similar affinity for fibrillar assemblies (FIG. 2). In contrast, although 6C3 does not demonstrate specificity for oligomers or fibrils by antigen/antibody blotting, standard ELISA shows 6C3 antibodies display some preference for oligomeric assemblies over that of fibrillar assemblies (FIG. 2).

### Example 4

### Western Blot Analysis Using 7A2 and 6C3 Antibodies

To further characterize the 7A2 and 6C3 antibodies, Western blot analysis was performed. Unaggregated (U), oligomeric (O), and fibrillar (F) amyloid β protein1-42 were run on 12% NUPAGE Bis-Tris gels under non-reducing conditions. The blots were probed with purified 6C3 (1:10,000), 6E10 (1:5000), or 7A2 (1:1000) monoclonal antibodies.

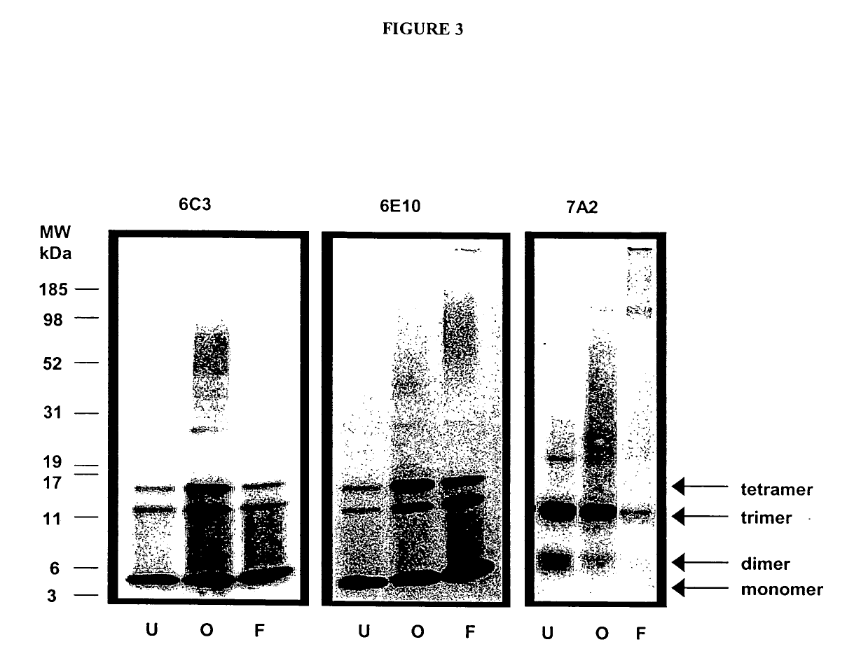

The oligomer-specific antibody (7A2) shows little recognition of fibrils by antigen/antibody blotting (FIG. 1) and ELISA (FIG. 2). By Western analysis of SDS-PAGE, 7A2 detects primarily dimer and trimer but no amyloid β protein monomers in unaggregated or oligomeric samples, and little immunoreactivity is detected in the fibril samples (FIG. 3). In contrast, 6C3 demonstrates a slight selectivity for oligomers over fibrils by ELISA (FIG. 2), however no differences are detected in Western blots between unaggregated, oligomer or fibril samples; here it reacts much like other amyloid β protein monoclonal antibodies such as 6E10 and 4G8 (FIG. 3).

### Example 5

### Bright Field Immunohistochemistry

The monoclonal antibodies 7A2 and 6C3 were analyzed on sections of human brain using standard peroxidase-based immunohistochemistry (FIG. 4). Tissue sections from the superior parietal lobule were obtained from well-characterized AD patients. The sections were stained with purified 6C3 antibody at a 1:20,000 dilution and with the tissue culture supernatant of 7A2 at a 1:1000 dilution. The magnification is 10×. In sections from AD brain, little 7A2 immunoreactivity with fibrillar amyloid plaques is detected (FIG. 4).

### Example 6

### Laser Scanning Confocal Microscopy (LSCM) of AD Brain Slices

In order to determine whether an antibody specific for soluble oligomers coexists in the same cells, plaques or areas in the neuropil as Thioflavin S-positive plaques, double or triple immunofluorescence assessment using LSCM is useful. Sections from the same brain regions as those studied using the peroxidase procedure above are used. In this procedure, the primary antibodies are each detected with a different fluorochrome (either directly conjugated to the primary antibody or to a species-specific or isoform-specific secondary antibody). The z-sections obtained from a series of confocal images can be stacked and both fluorescence channels combined following pseudo-coloring. These stacks can then be rotated to view the three-dimensional image from a number of angels. For double immunofluorescence confocal microscopy, the co-localization feature of the software Metamorph is used to establish the percent co-localization between two fluorochromes from representative digital images.

A problem frequently encountered in any immunofluorescence studies on aged human brains is the presence of significant amounts of autofluorescent lipofuscin that can be confused with the yellow fluorescence seen during colocalization. In order to minimize this problem, sections are placed in a potassium permanganate solution (0.25% in phosphate buffered saline) for 20 minutes, after which a brown color develops. The sections are washed in phosphate buffered saline for two minutes, and then treated with a solution of 1.0 g % potassium metabisulfite and 1.0 g % oxalic acid in phosphate buffered saline until the brown color dissipates, typically occurring in 1-6 minutes. Finally, the sections are washed three times in phosphate buffered saline for two minutes each. Alternatively, Cy 5, a fluorochrome that emits in the infrared range can be used, thereby circumventing the autofluorescence of lipofuscin that does not emit light in the infrared spectrum of light.

The monoclonal antibody 6C3 and an amyloid β protein polyclonal antibody (R1280) were used to immunohistochemically stain an amyloid plaque and diffuse amyloid β protein deposits in the temporal lobe from an AD patient. 6C3 detected diffuse amyloid plaque-like structures not detected by AD polyclonal antibodies (FIG. 5).

### Example 7

### Western Blot and Dot Blot Analysis Using 7A2 and 6C3 Antibodies

To still further characterize the 7A2 and 6C3 antibodies, Western blot analysis was repeated on unaggregated (U), oligomeric (O), and fibrillar (F) amyloid β protein1-42 as in Example 4. 1100 pMol amyloid β protein1-40 or 200 pMol amyloid β protein1-42 were run on 4-12% BIS-TRIS NuPAGE gels, transferred to PVDF membrane and probed with each respective antibody. In this experiment, the 7A2 antibody only detected oligomeric amyloid β protein1-42 assemblies (primarily dimer, tetramer and larger oligomers between approximately 27 and 44 kDa), whereas 6E10 and 6C3 detected multiple forms of amyloid β protein1-42 including monomer, trimer, tetramer and oligomers between approximately 27 and 80 kDa (FIG. 6). The data presented in FIG. 6 accurately reflects the identity of the bands. To further confirm the data, a range of antibody concentrations was hybridized to amyloid β protein1-42 transferred to PVDF. The oligomer specificity of 7A2 was retained over a wide range of antibody concentrations (FIG. 6). Note: “MOAB-1” and “MOAB-2” in FIG. 6 correspond to 7A2 and 6C3 antibodies, respectively.

Further confirmation of the specificity of 7A2 for oligomeric amyloid β protein1-42 was achieved using dot blot analysis of different conformations of amyloid β protein immobilized on nitrocellulose. In FIG. 7a, 10 pMol of amyloid β protein1-40, unaggregated amyloid β protein1-42, oligomeric amyloid β protein1-42, or fibrillar amyloid β protein1-42 were spotted on nitrocellulose and probed with 6E10, 6C3 or 7A2 antibodies. In FIG. 7b, a dilution series of amyloid β protein antigens was probed with 6E10, 6C3 or 7A2. In FIG. 7c, different ratios of oligomeric and fibrillar amyloid β protein1-42 were co-applied to nitrocellulose membrane and probed with 6E10, 6C3 or 7A2 antibodies. The dot blot analysis produced results consistent with both Western blot and ELISA results. 7A2 displayed greater affinity for oligomeric amyloid β protein1-42 as compared to amyloid β protein1-40, unaggregated or fibrillar amyloid β protein1-42. This selective high affinity interaction was maintained over a range of antigen concentrations. 6C3 and 6E10 were more immunoreactive to amyloid β protein1-42 than amyloid β protein1-40 but did not differentiate between oligomeric or fibrillar assemblies of amyloid β protein1-42. 7A2 also demonstrated specificity for oligomeric species in the presence of increasing concentrations of co-deposited fibrillar amyloid β protein1-42.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A composition comprising a purified monoclonal antibody that identifies soluble, non-fibrillar oligomeric amyloid β protein assemblies.

2. The composition of claim 1, wherein said amyloid β protein is the β1-42 protein.

3. A hybridoma that secretes said monoclonal antibody of claim 1.

4. A method for obtaining and isolating a hybridoma secreting said monoclonal antibody of claim 1, comprising:
a) providing spleen cells immunized with an antigen comprising soluble, non-fibrillar oligomeric amyloid β protein assemblies, wherein said antigen is recognized by said monoclonal antibody of claim 1;
b) fusing said immunized cells with myeloma cells under hybridoma-forming conditions; and
c) selecting those hybridomas that secrete monoclonal antibodies that specifically recognize assemblies comprising amyloid β proteins without recognizing fibrillar amyloid β protein assemblies.

5. A composition comprising a purified monoclonal antibody suitable for identification of fibrillar amyloid β protein assemblies that does not identify soluble, non-fibrillar oligomeric amyloid β protein assemblies.

6. The composition of claim 5, wherein said amyloid β protein is the β1-42 protein.

7. A hybridoma that secretes said monoclonal antibody of claim 5.

8. A method for obtaining and isolating a hybridoma secreting said monoclonal antibody of claim 5, comprising:
a) providing spleen cells immunized with an antigen comprising fibrillar amyloid β protein assemblies, wherein said antigen is recognized by said monoclonal antibody of claim 5;
b) fusing said immunized cells with myeloma cells under hybridoma-forming conditions; and
c) selecting those hybridomas that secrete monoclonal antibodies that specifically recognize assemblies containing amyloid β proteins without recognizing soluble, non-fibrillar oligomeric amyloid β protein assemblies.

9. A method for detecting at least one amyloid β protein assembly, comprising the steps of:
a) providing
i) a sample from a subject suspected of containing at least one amyloid β protein assembly; and
ii) an antibody that identifies amyloid β protein assemblies;
b) contacting said sample with said antibody under conditions such that said antibody binds to said amyloid β protein assembly, forming an antigen-antibody complex; and
c) detecting the presence of said antigen-antibody complex.

10. The method of claim 9, wherein said at least one amyloid β protein assembly comprises soluble, non-fibrillar oligomeric amyloid β protein comprising 2-12 amyloid β proteins.

11. The method of claim 9, wherein said at least one amyloid β protein assembly comprises fibrillar amyloid β protein comprising more than 12 amyloid β proteins.

12. The method of claim 9, wherein said sample is selected from the group consisting of blood, plasma, serum, serous fluid, and cerebrospinal fluid.

13. The method of claim 9, wherein said subject is selected from the group consisting of subjects displaying pathology resulting from Alzheimer's disease, subjects suspected of displaying pathology resulting from Alzheimer's disease, and subjects at risk of displaying pathology resulting from Alzheimer's disease.

14. The method of claim 9, further comprising the step of diagnosing Alzheimer's disease, wherein said Alzheimer's disease is selected from the group consisting of late onset Alzheimer's disease, early onset Alzheimer's disease, familial Alzheimer's disease and sporadic Alzheimer's disease.

15. The method of claim 9, wherein said detecting at least one amyloid β protein assembly comprises an enzyme-linked immunosorbent assay, wherein said enzyme-linked immunosorbent assay is selected from the group consisting of direct enzyme-linked immunosorbent assays, indirect enzyme-linked immunosorbent assays, direct sandwich enzyme-linked immunosorbent assays, indirect sandwich enzyme-linked immunosorbent assays, and competitive enzyme-linked immunosorbent assays.

16. The method of claim 9, further comprising the step of quantitating said at least one amyloid β protein assembly in said sample.

17. The method of claim 15, wherein said enzyme-linked immunosorbent assay further comprises an alkaline phosphatase amplification system.

18. The method of claim 15, further providing at least one capture antibody.

19. The method of claim 18, further providing at least one detection antibody.
